(19) United States
(12) Patent Application Publication
(10) Pub. No.: US 2011/0130786 A1
(43) Pub. Date: Jun. 2, 2011

(54) Method for Treating Headaches with Intra-Aural Devices

(75) Inventors: Lawrence G. Clayton, Rockford, IL (US); William R. Coole, Belvidere, IL (US)

(73) Assignee: Ascentia Health, Inc., Rockford, IL (US)

(21) Appl. No.: 12/628,581

(22) Filed: Dec. 1, 2009

(51) Int. Cl. A61B 17/00

(52) U.S. Cl. 606/204.15

(57) ABSTRACT

Methods for treating a headache of an individual with a pair of prosthetic intra-aural devices are provided. The methods includes inserting one of the intra-aural devices into each ear canal of the person. Later, the person may remove the intra-aural device from each ear canal. Each of the intra aural devices has a predefined shape substantially conforming to each ear canal, and is hollow in its interior to permit hearing, yet rigid enough to maintain its shape once inserted into the ear canal. The intra-aural device may be inserted at the onset of or prior to the symptoms of a headache, and may remain inserted until the headache lessens in severity, subsides or at any other time period. For migraine or other chronic headaches, the intra-aural devices may be worn to reduce at least one of the frequency, severity, and/or duration of the migraine headaches.

### FIELD OF THE INVENTION

The present invention relates generally to medical devices, and more particularly to medical devices for the reduction in severity, duration, frequency, and/or prevention and alleviation of headaches.

### BACKGROUND OF THE INVENTION

Many people suffer from headaches that are attributable to a variety of factors ranging from minor issues such as stress, eye strain, and menstrual cycles to severe disorders such as migraines. Headaches rank amongst the most common of local pain complaints, and may be frequent and reoccurring for many people. An individual suffering from a headache may experience minor to extreme pain depending upon the severity of the headache. The most common type of headache is referred to as a “tension type” or “muscle headache.” This type of headache accounts for more than seventy-five percent of all headaches.

Over the years, there have been a variety of methods devised to treat headaches, including the use of pharmaceutical compounds. Unfortunately, pharmaceutical compounds are not always effective in relieving certain types of headaches. In addition, many pharmaceutical products may have differing levels of efficacy for different people. Also, over time, patients may build up a resistance to the pharmaceutical compounds used in treatment, thus reducing their effectiveness or rendering them ineffective. Other patients may be allergic to the pharmaceutical compounds commonly used to treat headaches or develop ulcers due to the use of the compounds, and they are therefore unable to use pharmaceutical compounds at all.

In addition to pharmaceutical therapies, there have also been a number of invasive and non-invasive appliances devised to accomplish various headache treatment related goals. These appliances include devices designed to restrict blood flow to certain parts of the brain, or stimulate certain nerve endings. Invasive procedures typically include acupuncture or percutaneous electrical nerve stimulation (PENS). Both acupuncture and PENS can stimulate pain fibers at the site of application as well as within the region of the nerve being stimulated, thereby causing the patient greater pain than that already being endured due to the patient's headache. Although usually not as painful, non-invasive appliances are typically worn on the patient's head and resemble a head band. These non-invasive devices may apply a light pressure to the skull, or provide heating or cooling therapies. Many patients are deterred from wearing these non-invasive appliances in public due to a feeling of embarrassment or awkwardness due to the size and appearance of the appliances.

There exists, therefore, a need in the art for a discrete method of treating headaches that is not subject to the limitations of pharmaceutical therapies or invasive and noninvasive treatments, and which would not subject a patient to any further pain or emotional discomfort than already caused by the patient's headache. The method of treatment disclosed herein provides such a solution. These and other advantages of the disclosed methods of treatment will be apparent from the description provided.

### BRIEF SUMMARY OF THE INVENTION

In view of the above, embodiments of the present invention provide an innovative and effective method for treating certain types of headaches.

An embodiment of the invention provides a method of treating a headache of a person with a pair of intra-aural devices, one fitted to each of the left and right ear canals. The method according to this aspect includes the step of inserting the intra-aural devices into each of the ear canals of a person. This method can be performed by hand by the person using the intra-aural devices . The person using the intra-aural devices can insert the intra-aural devices prior to or after the onset of the symptoms of a headache. The method according to this aspect can also include the step of removing the intra-aural devices from the ear canals. As with insertion, removal can be done effortlessly by the person using the intra-aural devices. The person using the intra-aural devices can remove the intra-aural devices after symptoms of the headache have lessened in severity, subsided, or at some other time.

In another aspect, an embodiment of the invention provides a method for treating a headache of a person with a pair of intra-aural devices by inserting the intra-aural devices into the person's ear canals. The ear canal includes an isthmus and the ear includes a tragus and an inter-tragal notch. The intra-aural devices according to this embodiment include a hollow and substantially rigid structural body, each having a predefined shape substantially conforming to a contour of a portion of each individuals left and right ear canals based upon an impression procedure accomplished while the jaw is in an open position. The structural body extends approximately between the entrance to the ear canal and its isthmus. The structural body also includes a proximal base portion adapted for positioning the base portion adjacent to the entrance of the ear canal. The structural body further includes a distal end portion adapted to extend an effective distance into the ear canal, wherein a scalloped indenture extends away from a boundary edge of the proximal base portion and across a surface of the structural body. The scalloped indenture is adapted for positioning behind the tragus of the ear According to this aspect, removing the intra-aural device includes grasping the post element of the intra-aural device and pulling upon the post element in a direction opposite to the direction of insertion into the ear canal.

In yet another aspect, an embodiment of the invention provides a method for treating a headache of a person, including the step of inserting the intra-aural devices into the ear canals of the person. This method is performed by hand by the wearer of the intra-aural devices. The individual using the intra-aural devices can insert the intra-aural devices prior to or after the onset of symptoms of a headache. The method according to this aspect can also include the step of removing the intra-aural devices from the ear canals. Removal is accomplished by the person using the intra-aural devices retracting each prosthetic device by use of their removal posts. The person using the intra-aural devices can remove the intra-aural devices after symptoms of the headache have lessened in severity, subsided, or at some other time.

In still yet another aspect, an embodiment of the invention provides a method for treating migraine headaches with a pair of intra-aural devices, one fitted to each of the left and right ear canals. The method according to this aspect includes the step of inserting the intra-aural devices into each of the ear canals of a person. This method can be performed by hand by the person using the intra-aural devices. The person using the intra-aural devices can insert the intra-aural devices prior to the onset of the symptoms of the migraine headache to reduce the frequency, severity, and/or duration of the migraine headaches, or after the onset of the symptoms to reduce at least the severity and/or duration of the migraine headache. The method according to this aspect can also include the step of removing the intra-aural devices from the ear canals. As with insertion, removal can be done effortlessly by the person using the intra-aural devices. The person using the intra-aural devices can remove the intra-aural devices after symptoms of the migraine headache have lessened in severity, subsided, or at some other time.

Other embodiments of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is an isometric view of an exemplary intra-aural device for insertion into an ear canal for the treatment of headaches;

FIG. 2 is a view illustrating an outer ear; and

FIG. 3 is cut-away side view of the intra-aural device of FIG. 1 inserted into an ear canal;

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Exemplary constructions and practices will now be described through reference to the drawings, wherein like elements are designated by like reference numerals in the various views. A method for treating headaches with a pair of intra-aural devices is disclosed herein. In the following, one of the pair of intra-aural devices is structurally defined, followed by a description of the method for using the pair of intra-aural devices to treat and/or prevent headaches. Also, as used herein, treating a headache includes one or more of reducing the frequency of the onset of headaches, reducing the severity of the symptoms of the headache, reducing the duration of the symptoms of the headache, and/or preventing the onset of symptoms of the headache.

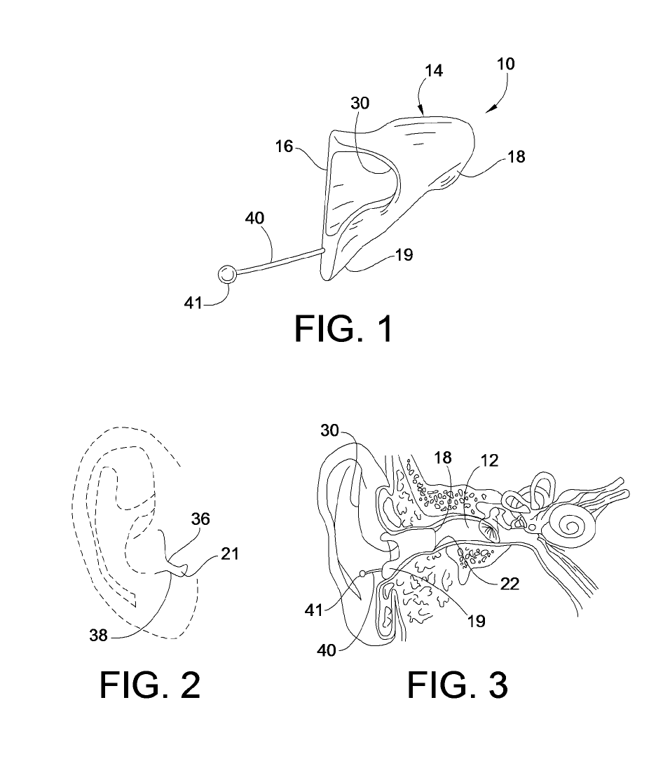

For purposes of illustration, FIG. 1 illustrates one of the pair of intra-aural devices 10 adapted for insertion into an ear canal 12 as shown generally in FIG. 3. According to one embodiment of a contemplated practice, the intra-aural device 10 includes a hollow structural body portion 14 molded from a substantially rigid material such as acrylic or the like. Accordingly, the structural body portion 14 is substantially incompressible and maintains its shape upon insertion

In the illustrated construction, the structural body portion 14 is substantially hollow along its length to avoid interference with hearing and facilitate sound transmission.

In one embodiment, the structural body portion 14 is individually fitted to the user as will be described below. In other embodiments, the structural body portion 14 is mass produced to have a universal fit corresponding substantially to the individual shaped ear canals 12 or various ear sizes of users or in varying sizes. Thus, following insertion of the intra-aural device 10, the structural body portion 14 is in substantially conforming relation relative to contours at the inner surface of the ear canal 12 such that the intra-aural device 10 is held in a substantially nested relation relative to the interior surface of ear canal 12. If desired, one or more additional compressible cushioning layers (not shown) may be provided around at least a portion of the structural body portion 14. However, such a cushioning layer is in no way essential. By way of example only, and not limitation, materials for forming such a cushioning layer may include PVC, silicone or the like.

In other embodiments of the present invention, the structural body portion 14 utilizes an expandable shell or endoskeleton structures embedded within such expandable materials. One such embodiment utilizes a stent (not shown) embedded in the structural body portion 14. The stent has a smaller diameter at a temperature below the temperature within the ear canal and is expandable to a greater diameter when subjected to the user's body temperature within the ear canal of the user. As the stent expands within the structural body portion 14, it provides a contoured fit to the interior wall of the external ear canal, thereby providing a universal fit. Exemplary materials and stent configurations are described in US Patent Application Publication No. US 2007/0183613 A1, entitled Self Forming In-The-Ear Hearing Aid with Conical Stent, to Juneau et al., the teachings and disclosure of which are hereby incorporated in their entireties by reference thereto. Another configuration providing conformance to the ear canal once inserted based on a wearer's body heat is described in U.S. application Ser. No. 12/561,464, entitled Ear Insert for Relief of TMS Discomfort and Headaches, the teachings and disclosure of which are also hereby incorporated in their entireties by reference thereto.

As will be described in more detail below, although described above as generally having a structural body portion 14 that is in substantially conforming relation relative to the contours at the inner surface of ear canal 12 of various users due to its mass production, the intra-aural device 10 is preferably manufactured specific to a person's ear canals by taking individual measurements via impressions of the ear canals with the jaw in a measured open condition 12 prior to manufacturing the intra-aural device 10. In this regard, the structural body portion 14 will provide a custom fit for a given patient as opposed to being manufactured to conform to a typical ear canal 12.

As shown in the illustrated construction, structural body portion 14 includes a proximal base portion 16 and a distal end portion 18. The proximal base portion 16 is of greater diameter than the distal end portion 18. As best illustrated in FIG. 3, proximal base portion 16 (See FIG. 1) is adapted to be positioned in inserted relation substantially adjacent to the opening of the ear canal 12. In the illustrated construction, proximal base portion 16 includes an extended peninsular leg portion 19 adapted to project generally downwardly when structural body portion 14 is in inserted position within the ear canal 12. In this regard, peninsular leg portion 19 projects into the inter-tragal notch 21 shown in FIG. 2 adjacent to the tragus 36 and the opposing anti-tragus 38 portions of the outer ear. Distal end portion 18 of structural body portion 14 is adapted to extend approximately to the bend in the ear canal known as the isthmus 22. The isthmus 22 is located approximately 20-23 millimeters from the outside of an adult ear and is in close proximity to the temporo-mandibular joint.

As best illustrated through simultaneous reference to FIGS. 1 and 3, according to the illustrated construction, a scalloped indenture 30 extends away from an edge of the proximal base portion 16. In this regard, it is contemplated that the scalloped indenture 30 may be formed by any suitable technique. By way of example only, and not limitation, one such technique involves post-formation sculpting using cutting tools or the like although other techniques may be used as desired. As shown, the scalloped indenture 30 extends generally towards distal end portion 18 and is positioned across a surface of structural body portion 14 adapted to reside immediately behind the tragus 36 and opposing anti-tragus 38 of the outer ear. It is contemplated that the scalloped indenture 30 may have a length and width suitable to accept substantially the entire lobe of tragus 36 thereby facilitating the ability of tragus 36 to bend inwardly towards the opening of the ear canal 12 substantially without obstruction thereby reducing or avoiding compression of tragus 36 during use. It has been found that structural body portion 14 incorporating such a scalloped indenture 30 maintains its structural integrity as described hereinafter despite the reduction in supporting material.

According to the illustrated construction, it is contemplated that at least one anterior projecting post element 40 may extend away from a surface of peninsular leg portion 19. As shown, post element 40 projects in a direction extending generally away from distal end portion 18 so as to project towards the exterior of the ear. Following insertion, at least a portion of post element 40 may reside outside of the ear canal 12 at a position within the vicinity of inter-tragal notch 21. In this position a wearer may grasp post element 40 to facilitate removal of the device. Post element 40 is preferably substantially pliable to facilitate grasping. At the same time, post element 40 should be characterized by sufficient strength to avoid breakage. By way of example only, and not limitation, it has been found that a suitable post element 40 may be formed from monofilament nylon adhesively bonded onto a surface of peninsular leg portion 19. However, other suitable polymeric or non-polymeric materials may likewise be utilized.

As shown, post element 40 may include a bulbous head portion 41. Such a bulbous head portion 41 may enhance the ability of a wearer to grasp the post element 40 during removal of the intra-aural device 10 from the ear canal 12. In the event that post element 40 is formed from nylon or other thermoplastic material, a suitable bulbous head portion 41 may be formed by selectively melting the terminal end of post element 40 to form a melted polymer bead which is thereafter permitted to re-solidify.

An intra-aural device 10 has been introduced and described in structural terms in the foregoing. A method for using a pair of such intra-aural devices 10 to treat and/or prevent headaches will now be described in detail in the following.

Referring now to FIGS. 1, 2, and 3, a method for treating a headache of a person with a pair of the intra-aural devices 10 as described herein includes the step of inserting one intra-aural device 10 into each of the ear canals 12 of the person. As noted above, when the intra-aural device 10 is fully inserted, the distal end portion 18 of the structural body portion 14 extends an effective distance into the ear canal, which is approximately to the bend in the ear canal 12 known as the isthmus 22 (See FIG. 3). Although the intra-aural device 10 is contoured to fit the ear canal 12 when the jaw is in an opened position, it does not prohibit normal jaw functionality, e.g. mastication and audible communication, once inserted.

In one embodiment, a person having a headache inserts the intra-aural devices 10 after the onset of symptoms of a headache. However in another embodiment, the person inserts the intra-aural devices 10 prior to detecting any symptoms of a headache. As such, the intra-aural devices 10 function as a preventive measure, thereby deterring the onset of the symptoms of a headache, reducing the frequency of the onset of the symptoms of a headache, e.g. a migraine headache, and/or preventing the onset of the symptoms of a headache. When used in this embodiment, the person continues to wear the intra-aural devices after the onset of symptoms so as to lessen the severity of the symptoms of the headache and/or to lessen the duration of the symptoms of the headache.

In one embodiment, once the person has inserted the intra-aural devices 10, the person leaves each of the intra-aural devices 10 inserted in the ear canal 12 until the symptoms of their headache have lessened in severity, subsided or at some other time. In another embodiment however, the person leaves each of the intra-aural devices 10 inserted for a predetermined period of time, regardless of the continued presence or lack thereof of headache symptoms. Additionally, a person can leave the intra-aural devices 10 inserted indefinitely if, for example, the person suffers from chronic headaches, such as, e.g., migraines, so as to at least one of reduce the frequency of such headaches, reduce the severity of the symptoms of such headaches, and/or reduce the durations of such headaches once they occur.

Once the person has inserted each of the intra-aural devices 10, the person may later remove them in substantially the same manner as inserted. To remove either of the intra-aural devices 10, the person grasps and pulls upon the post element 40 in a direction opposite the direction of insertion into the ear canal 12. Although described above as incorporating the post element 40 and head portion 41, the intra-aural device 10 can also be removed in a like manner when the post element 40 and head portion 41 are omitted.

In one embodiment, the method as described above also includes the step of first taking measurements of each of the individual's ear canal dimensions via a scanning device or other type of technology. Specifically, the dimensions of a particular individual's ear canals in the jaw opened position using the spacer 12 are determined via a jaw open position using a spacer or bite block between the midline of the individual's upper and lower teeth. Thereafter, the step of manufacturing the intra-aural devices 10 to have a structural body portion 14 that is contoured to correspond to the person's particular ear canals 12. Alternatively, the step of first taking measurements of a particular person's ear canals by way of a scanning technique or other technology 12 is omitted, and instead an impression is made by way of a standard impression technique of the person's ear canals 12. A custom sized intra-aural 10 device is then manufactured using a negative of each of the impressions made from the person's ear canals 12. Additionally, measurements may be taken from the impressions made from the person's ear canals, and a custom sized intra-aural device can then be manufactured using a process such as stereolithography.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar references in the context of describing the invention (especially in the context of the following claims) is to be construed to cover both the singular and the plural, unless otherwise indicated herein or clearly contradicted by the context in which used. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by the context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by the context in which used.

What is claimed is:

1. A method for treating a headache of a person, comprising the step of inserting an intra-aural device into each ear canal of the person.

2. The method of claim 1, wherein the step of inserting the intra-aural device comprises the step of inserting the intra-aural device into each ear canal of the person after the onset of symptoms of the headache.

3. The method of claim 1, wherein the step of inserting the intra-aural device comprises the step of inserting the intra-aural device into each ear canal of the person prior to the onset of symptoms of the headache.

4. The method of claim 1, wherein the step of inserting the intra-aural device comprises the step of inserting each of the intra-aural devices such that the intra-aural device extends between an opening of the ear canal to an isthmus of the ear canal.

5. The method of claim 1, further comprising the step of removing the intra-aural device from each ear canal after inserting the intra-aural device into each ear canal.

6. The method of claim 5, wherein the step of removing the intra-aural device from each ear canal comprises the step of removing the intra-aural device from each ear canal after one or more of symptoms of the headache have lessened in severity, the symptoms of the headache have subsided, or after a predetermined time.

7. A method for treating a headache of a person, comprising the step of inserting an intra-aural device into each ear canal of the person, the ear having a tragus and an inter-tragal notch, wherein the ear canal includes an isthmus, the intra-aural device comprising: a hollow and substantially rigid structural body having a predefined shape substantially conforming to a contour of a portion of the ear canal, the structural body extending approximately between the entrance to the ear canal and the isthmus when the intra-aural device is inserted in said ear canal, the structural body including a proximal base portion having an extended peninsular leg portion which extends substantially to the inter-tragal notch, the proximal base portion adapted for positioning adjacent to the entrance of the ear canal, the structural body further including a distal end portion adapted to extend an effective distance into the ear canal, wherein a scalloped indenture extends away from a boundary edge of the proximal base portion across a surface of the structural body, the scalloped portion adapted for positioning behind the tragus of the ear.

8. The method of claim 7, further comprising the steps of:
removing the intra-aural device from each ear canal by grasping a post element of the intra-aural device and pulling upon the post element in a direction away from the ear canal, wherein the post element projects away from the proximal base portion, the post element extending to a position outside of the ear canal when the intra-aural device is inserted within the ear canal.

9. The method of claim 7, further comprising the steps of:
taking measurements of an ear canal with the person's mouth in an open position; and
manufacturing the intra-aural device to have a contoured surface corresponding to the measurements.

10. The method of claim 7, further comprising the steps of:
making impressions of ear canals with the person's mouth in an open position; and
manufacturing the intra-aural device to have a contoured surface corresponding to a negative of the impressions.

11. A method for treating a headache of a person, the headache not related to a temporo-mandibular joint disorder, by using an intra-aural device, comprising the step of inserting the intra-aural device into each ear canal of the person.

12. The method of claim 11, wherein the step of inserting the intra-aural device comprises the step of inserting the intra-aural device into each ear canal after the onset of symptoms of a headache.

13. The method of claim 11, wherein the step of inserting the intra-aural device comprises the step of inserting the intra-aural device into each ear canal prior to the onset of symptoms of a headache.

14. The method of claim 11, further comprising the step of removing the intra-aural device from each ear canal.

15. The method of claim 14, further comprising the step of waiting for symptoms of the headache to lessen in severity or to subside prior to removing the intra-aural device from each ear canal.

16. A method for treating a migraine headache of a person by using an intra-aural device, comprising the step of inserting the intra-aural device into each ear canal of the person.

17. The method of claim 16, wherein the step of inserting the intra-aural device comprises the step of inserting the intra-aural device into each ear canal after an onset of symptoms of the migraine headache to lessen at least one of a severity of the migraine headache or a duration of the migraine headache.

18. The method of claim 16, wherein the step of inserting the intra-aural device comprises the step of inserting the intra-aural device into each ear canal prior to an onset of symptoms of the migraine headache to reduce at least one of a frequency of occurrence of the migraine headache, a severity of the migraine headache, or a duration of the migraine headache.

19. The method of claim 16, further comprising the step of removing the intra-aural device from each ear canal.

20. The method of claim 19, further comprising the step of waiting for symptoms of the migraine headache to lessen in severity or to subside prior to removing the intra-aural device from each ear canal.
